(19) United States
(12) Patent Application Publication
(10) Pub. No.: US 2021/0151151 A1
(43) Pub. Date: May 20, 2021

(54) MEDICAL INFORMATION QUERY AND INPUT SYSTEM, MEDICAL INFORMATION QUERY AND INPUT METHOD, AND PROGRAM FOR PERFORMING SAME

(71) Applicant: HD JUNCTION, INC, Seoul (KR)

(72) Inventors: Jee Hyun SHIN, Seoul (KR); Jae Hyung HAN, Seongnam (KR)

(21) Appl. No.: 17/263,325

(22) PCT Filed: Jul. 29, 2019
(86) PCT No.: PCT/KR2019/009384

§ 371 Date: Jan. 26, 2021

(30) Foreign Application Priority Data
Jul. 27, 2018 (KR) 10-2018-0088179
Aug. 17, 2018 (KR) 10-2018-0096205

(51) Int. Cl. G16H 10/60; G16H 15/00; G16H 20/00

(52) U.S. Cl. CPC G16H 10/60; G16H 20/00; G16H 15/00

(57) ABSTRACT

The present invention relates to a medical information query and input method, and a program and a system for performing same. Provided are a medical information query and input method and a program and a system for performing same, the medical information query and input method including, a step for retrieving, from a database, medical information about a patient before a reference date; a step for generating, from the patient's medical information, an object for each date according to a set field item; and a step for displaying, on an interface, each of the objects in a separate section format, by date in a time series in the corresponding fields.

### TECHNICAL FIELD

The present invention relates to a medical information query and input method and a program for performing the same, and more specifically, to a medical information query and input system, a query and input method, and a program for performing the same for improving readability and user convenience.

### BACKGROUND ART

While medical information about patients was conventionally managed by being written on paper, computerization has been achieved in the form of an electronic medical record at present. A system for querying, recording and using such an electronic medical record is constructed in hospitals, and a user accesses the electronic medical record through a user terminal such as a computer to query and record the electronic medical record.

However, an interface of a conventional system for querying and inputting electronic medical records is configured in the form of a library, and thus there is a problem that a procedure for accessing a desired record is cumbersome. In addition, since medical information is displayed through different windows according to dates or medical information types, it is difficult for a user to check a time-series change in a record and give medical treatment. There is a limit to access and use such a conventional system interface by an unprofessional user such as a patient due to a complicated structure thereof and there is a problem of deterioration of adaptability to user terminals diversified into tablet PCs, mobile phones, and the like.

### DISCLOSURE

### [Technical Problem]

To solve the aforementioned problems, the present invention provides a medical information query and input system, a query and input method, and a program for performing the same which can easily query desired medical information of a user and check a medical history and a state of a patient in a time series.

### [Technical Solution]

To accomplish the object, the present invention provides a medical information query and input method including: retrieving, from a database, medical information about a patient before a reference date; generating, from the medical information about the patient, objects for each date according to a set field item; and displaying, on an interface, each of the objects in a separate section format, by date in a time series in a corresponding field.

In addition, the present invention may further include inputting details of a field corresponding to the reference date or a date after the reference date using a section displayed on the interface prior to the reference date.

Here, the inputting may include selecting an already displayed section of the corresponding field and moving the section through a method of dragging the section to a position of the reference date or a position after the reference date to generate a new section at the corresponding position. Specifically, the inputting may include: generating a new section by dragging a section of a predetermined field to be input before the reference date to the position of the reference date; and generating medical information corresponding to the predetermined field of the reference date.

Here, the generating of the medical information may include selecting the generated section by a user to call an input window and correcting medical information of an existing section displayed through the input window to generate the medical information corresponding to the predetermined field of the reference date.

Furthermore, the generating of the medical information may include regenerating an object in which details corrected by the user have been reflected and displaying the regenerated object M the generated section. Here, the predetermined field may be one of a diagnosis field, a medical record field, and a prescription field.

Further, the inputting may include: generating a new section by dragging a section of a predetermined field to be reserved at a position corresponding to the reference date or before the reference date to the position of the reference date or a position after the reference date; and generating reservation information corresponding to the predetermined field after the reference date.

Here, the generating of the reservation information may include selecting the generated section by a user and inputting a reservation date by the user. In addition, the method may further include transmitting the generated reservation information to a prescription transfer system. Here, the predetermined field may be one of an examination field and a prescription field.

Further, when an examination result corresponding to the reserved information does not exist in the medial information about the patient transmitted from the database, the nation result may be displayed through the generated section using a predefined method such that a user can recognize that the examination result does not exist.

Further, when a user selects a position at which a section is not displayed on the interface, an input window corresponding to a corresponding field of a corresponding date may be displayed, and a new section in which an object corresponding to details input by the user through the input window is displayed may be generated at the position.

Furthermore, when the user selects the section, detailed information corresponding to the section may be displayed through an additional detailed display window. Further, when the user selects a specific field, only medical information corresponding to the selected field may be extracted and displayed through an additional window in a time series.

In addition, to accomplish the object of the present invention, the present invention may provide a medical information query and input program executing: retrieving, from a database, medical information about a patient before a reference date; generating, from the medical information about the patient, objects corresponding to set time axis information and set fields; and displaying, on an interface, each of the objects in a separate section format in a time series according to a corresponding field.

In addition, the present invention may provide a medical information query and input system including: an interface for displaying medical information about a patient in separate section formats according to date information: and field information and allowing a user to input medical information about a patient; and a processor for processing medical information about a patient provided from a database such that the medical information is able to be displayed on the interface and processing information input through the interface, wherein the processor includes: a data transfer unit for transmitting/receiving medical information about a patient to/from a database; an object generation unit for generating, from medical information about a patient provided from the database, each object corresponding to set time axis information and a set field such that the object is able to be displayed in a section on the interface: and an input processing unit for processing information input by a user through the interface.

### [Advantageous Effects]

According to the present invention, a user can easily ascertain temporal changes iii related information and correlation between pieces of field information because medical information of multiple dates can be simultaneously checked through one interface screen in a time series.

Furthermore, a display on an interface has a structure similar to a conventional paper medical chart in the present invention, and thus a user-familiar use environment can be provided and a user can easily identify information for each field through the display.

In addition, since new input can be performed using existing patient medical information according to the present invention, information can be easily input and the present invention can be easily used in various device environments such as a touchscreen.

### DESCRIPTION OF DRAWINGS

FIG. 1 is a diagram illustrating a configuration of a medical information query and input system according to an embodiment of the present invention.

FIG. 2 is a block diagram illustrating major components of a user terminal of FIG. 1.

FIG. 3 is a diagram illustrating an interface of FIG. 2.

FIG. 4 is a diagram illustrating a display of details of some sections in FIG. 3.

FIG. 5 is a diagram illustrating a time series display of some examination results through an additional window in FIG. 3.

FIG. 6 is a flowchart illustrating a medical information query and input method performed by the system of FIG. 1.

FIG. 7 is a diagram illustrating an example of inputting details of a first field to an interface.

FIG. 8 is a diagram illustrating another example of inputting details of the first field to the interface.

FIG. 9 is a diagram illustrating an example of inputting details of a second field to the interface.

FIG. 10 is a diagram illustrating display formats according to scales for each field or subfield.

FIG. 11 is a flowchart illustrating an interface display method according to time axis adjustment.

FIG. 12 is a diagram illustrating an interface in a state in which a first query period is set.

FIG. 13 is a diagram illustrating the interface in a state in which a second query period is set.

FIG. 14 is a diagram illustrating the interface in a state in which a third query period is set.

FIG. 15 is a diagram illustrating the interface in which information relevant to a representative value selected by a user is displayed.

FIG. 16 is a flowchart illustrating a method of selectively displaying information related to a representative value selected by a user.

### MODE FOR DISCLOSURE

Hereinafter, a medical information query and input system, a query and input method, and a program for performing the same according to embodiments of the present invention will be described in detail with reference to the drawings. In the following description, positional relation of components will be described on the basis of the drawings in principle. In addition, in the drawings, structures of the invention may be simplified or exaggerated as necessary for convenience. Accordingly, the present invention is not limited thereto and various devices may be added, modified, or omitted.

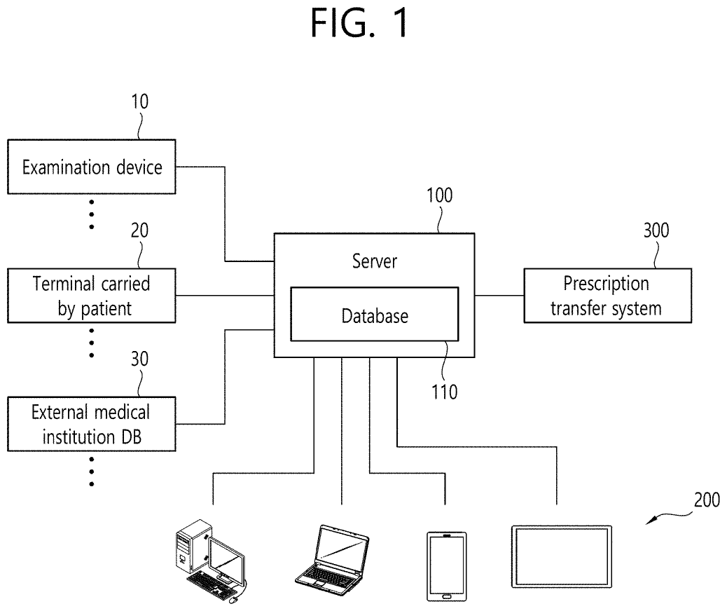

FIG. 1 is a diagram illustrating a configuration of a medical information query and input system according to an embodiment of the present invention. As illustrated in FIG. 1, a server 100 is configured to be connected to various examination devices 10, various terminals 20 carried by patients, servers 30 of external medical institutions, and a plurality of user terminals 200 using a network to receive information. This server 100 includes a database 110 for storing various types of medical information and stores various types of medical information about patients provided through the network in the database 110. Data stored in the database 110 may include various types of information about personal information and past medical histories of patients. For example, the data may include information personally recorded by medical personnel, such as medical records, operation records, nursing records, and anesthesia records. Alternatively, the data may include result information about various examinations such as image examination and functional examination, and information about details of treatment and details of prescription performed at the time of past medical treatment. Furthermore, the data may include medical information about patients received from external medical institutions and information about various measurement results and request details transmitted through devices carried by patients.

Although FIG. 1 illustrates a structure in which the server 100 is connected to the plurality of user terminals 200, the server may be connected to a single user terminal in a small medical institution such as a private hospital. Furthermore, although FIG. 1 illustrates a configuration in which the server and the user terminals are separate devices, a user terminal may include a database so that it can serve as both a server and a user terminal. Although such a server can be configured as hardware included in a medical institution, the server may be configured using a cloud server on the Internet such that a user can access it anywhere through the Internet.

The user terminals 200 are devices by which users can query medical information about patients and input medical information through an interface. These user terminals 200 can be configured using various electronic devices by which information can be displayed to users and users can input information, such as a desktop computer, a notebook computer, a tablet PC, and a mobile phone. Although the user terminals 200 are user terminals used by medical personnel in the description of the present embodiment, the present invention is not limited thereto and they may be configured as user terminals by which patients can query their medical record information and input information.

Medical information about patients input through the user terminals 200 can be newly stored or upgraded in the database 110. Further, when order information such as medical treatment details, prescription details, and reservation is input through the user terminal 200, the corresponding information can be transmitted to a prescription transfer system 300 through the server 100 such that the corresponding order can be performed.

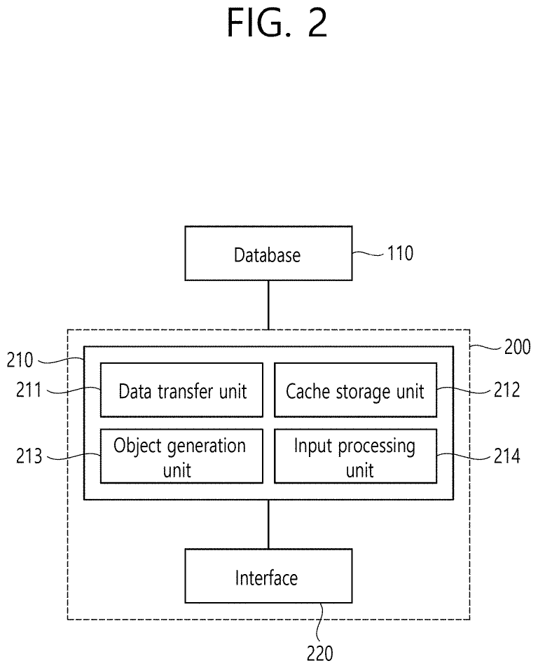

FIG. 2 is a block diagram illustrating major components of the user terminal of FIG. 1. As illustrated in FIG. 2, the user terminal 200 may include a processor 210 and the interface 220.

The processor 210 is a component that sends a request for medical information about a patient requested through the interface 220 to the database 110 and processes the medical information about the patient provided through the database 110 such that the medical information can be displayed through the interface 220. Here, the database 110 is indicated using the reference sign of the database of the server for convenience but it is not limited thereto and may be a database of another external system or a database included in the user terminal. In addition, the processor 210 is a component that transmits input information input through the interface 220 such that the input information can be stored in the database 110 and generates and transfers order information such that order details among the input information can be transmitted to the prescription transfer system 300.

In addition, the interface 220 is a component that displays medical information to a user through a display of the user terminal 200. Further, the user can input information such as a medical record, medical treatment details, and prescription details through the interface 220. Here, the user can input various types of information through the interface using a keyboard, a mouse, an input pen, or the like, and if the display is configured as a touchscreen, may input information through touch.

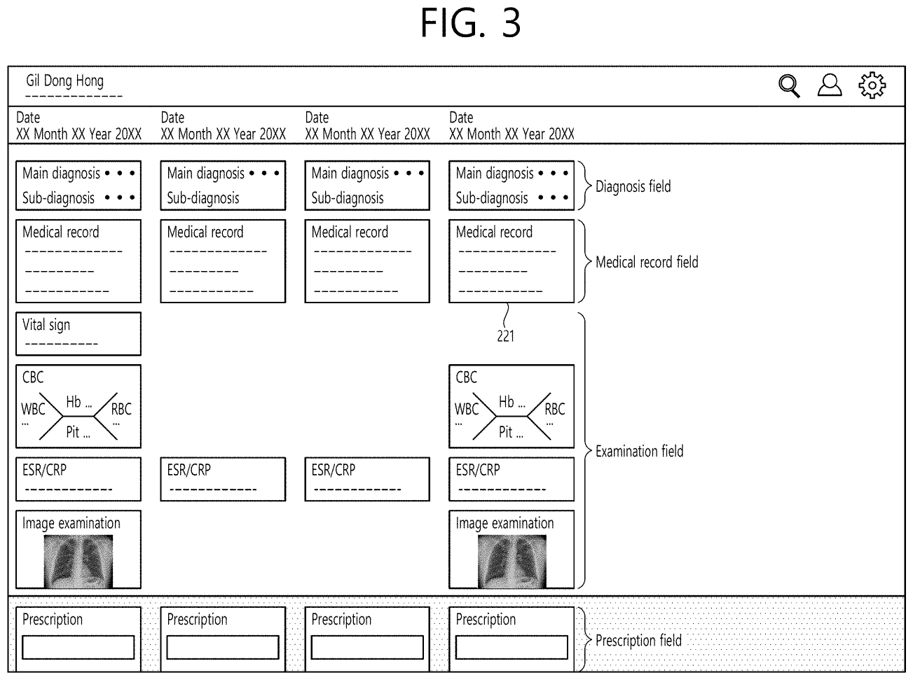

FIG. 3 is a diagram illustrating the interface of FIG. 2. As illustrated in FIG. 3, patient information such as the name, sex, and age of a patient is displayed and selection icons through which various functions can be selected are disposed at the top of the interface 220. In addition, a space in which various types of medical information of a selected patient are displayed or recorded is formed in the lower part. Such medical information about a patient includes various types of medical history information such as a diagnosis record, an examination record, an operation record, and a hospitalization record. This medical information is displayed in such a manner that events corresponding to a medical history are displayed by dates along a time axis in the horizontal direction, as illustrated in FIG. 3. Accordingly, a user can check the medical history of the patient through one interface screen in a time series.

Specifically, dates on which events corresponding to the medical history of the patient occur are displayed along the time axis. In general, a reference date is displayed at a position a predetermined distance from the left side of the time axis and dates prior to the reference date are displayed on the left of the reference date. Here, the reference date may be a set date and is generally the day when a user queries or inputs medical information. In addition, medical information corresponding to each date is displayed under each date in the horizontal direction. Here, the vertical axis is divided into a plurality of fields according to details and characteristics of medical information. These fields may include a diagnosis field, a medical record field, an examination field, a prescription field, and the like. Accordingly, medical information about patients stored in the database is classified according to characteristics thereof and displayed at positions of corresponding fields by dates corresponding to medical histories on the interface. A user can check details corresponding to each field while scrolling medical information by dates in the vertical direction. However, some fields (the prescription field in the present embodiment) may be configured to selectively rise up from the bottom of the interface in consideration of user convenience, as illustrated in FIG. 3 (here, a field is a superordinate concept including a set of subfields and is used as a term discriminated from a subfield but it means classification according to information characteristics and may be used as a term including a subfield itself).

In an interface of a conventional system, a procedure for accessing information through the interface is cumbersome because the interface is configured in the form of a library and it is difficult for a user to perform synthetic determination based on past medical information about a patient because information is individually managed and displayed. On the other hand, according to the present invention, a user can simultaneously check medical information of multiple dates through one interface screen in a time series. Furthermore, it is easy to ascertain temporal changes in related information because medical information about a patient is classified by field and displayed in a time series. In particular, since pieces of information corresponding to various fields are displayed on one screen, a correlation between pieces of information of fields can be easily ascertained (for example, a correlation between blood sugar test level changes according to a prescribed medicine change). Accordingly, a user can easily ascertain a medical history of a patient and details of prescription through the interface according: to the present invention and synthetically determine changes in a state of the patient and effectiveness of previous prescription.

Patient information by dates and by fields is provided in the form of a section on the interface. Here, provision of a section form means that each piece of information is formed as a separate entity and displayed on the interface such that it can be graphically discriminated from other pieces of information by dates and by fields. In addition, a user can view details of information in units of section and move a position of information on the interface in units of section. For example, information corresponding to each date and each field is displayed as a section 221 in a card shape on a background of the interface, as illustrated in FIG. 3. This structure is similar to a conventional paper medical chart and thus provides a user-familiar use environment, and a user can easily identify information corresponding to each field. However, one section 221 is not configured for each field of a corresponding date, and when a plurality of subfields is present for a field, separate sections are configured for the respective subfields. For example, separate sections can be displayed according to examination types such as blood examination and X-ray examination in the examination field, and separate sections can be displayed according to prescription types such as prescription of medicine, prescription of medical treatment, prescription of examination, and prescription of rehabilitation in the prescription field.

In this manner, a default screen of the interface according, to the present embodiment is displayed such that sections are aligned by dates and by fields. Accordingly, there is no information corresponding to some fields on a specific date, and thus, a blank part in which a section is not generated may be generated. Therefore, the interface includes a function of aligning and displaying sections upward such that a blank is not generated between sections of the same date. Accordingly, a user can check medical information about a patient while minimizing scrolling of the screen in the vertical direction by selecting the function in query of records by dates.

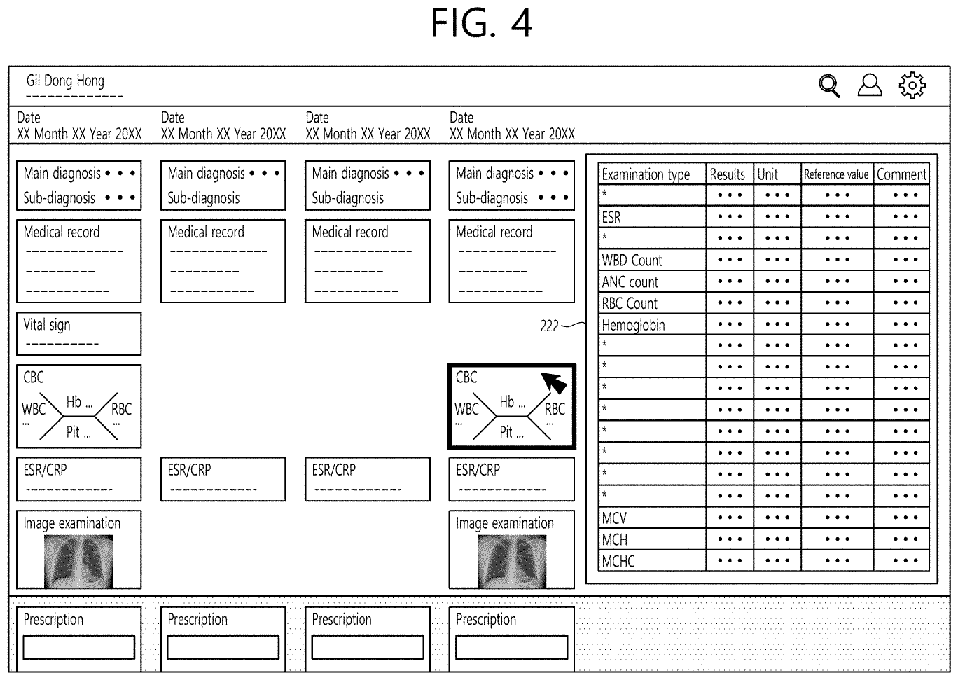

FIG. 4 is a diagram illustrating a display of details of some sections in FIG. 3. In each section 221 displayed on the interface 220, information of a corresponding date and a corresponding field is processed and displayed. When a user requests query of detailed information of the corresponding section, the detailed information is displayed through an additional detailed query window 222. The request may be performed in such a manner that the user selects the corresponding section, and this selection may be performed through a clicking operation using a mouse, a touch operation through a touchscreen, or the like. The detailed query window 222 may be configured as a separate section generated on one side of medical information about a patient, as illustrated in FIG. 4. Since the detailed query window 222 is overlaid on the default display screen without an additional screen change operation, continuity of the medical information query operation of the user can be maintained.

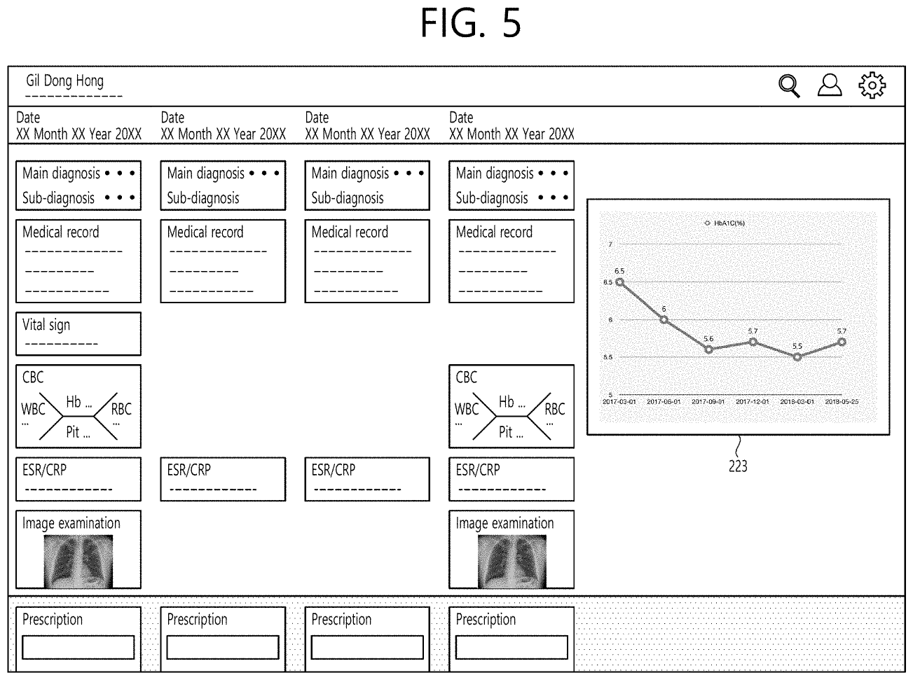

FIG. 5 is a diagram illustrating a time series display of some examination results through an additional window in FIG. 3. As described above, the interface of the present embodiment provides medical information by dates and by fields, and thus a user can check changes in each piece of field information in a time series through the default screen of the interface. However, the interface according to the present embodiment may include a function of displaying such changes in more detail through an additional detailed information window 223 for each field. When a user selects a field (e.g., X-ray examination result field) (e.g., clicks a field name), medical information corresponding to the field can be displayed through an additional detailed query window in a time series. In a case where query is performed through the default screen, some sections are displayed as blanks when the corresponding examination was not performed on some dates. However, in a case where information is displayed through an additional detailed query window, corresponding field information can be checked in detail without a blank. Furthermore, more detailed information than content displayed through each section can be displayed through a detailed query window, or changes over time can be displayed as a graph or can be processed into statistics and displayed if corresponding information pertains to numerical values.

A basic display form of the interface according to the present embodiment has been described above in detail with reference to FIGS. 3 and 5. However, the number of dates, time axis information such as displayed date periods, field types, and the like displayed on the interface may be changed according to user settings.

Referring back to FIG. 2, the configuration of the processor is described in m re detail. As illustrated in FIG. 2, the processor 210 may include a data transfer unit 211, a cache storage unit 212, an object generation unit 213, and an input processing unit 214.

The data transfer unit 211 is a component that is connected to various databases (including databases in user terminals, servers, additional examination devices, webs, etc.) in a wired/wireless manner and transmits and receives necessary information. Accordingly, when a user requests medical information about a specific patient through the interface 220, the data transfer unit 211 of the processor 210 receives the information from the database 110. Then, the data transfer unit 211 transmits the medical information input through the interface 220 such that the medical information can be stored in the database 110. Furthermore, when the user inputs a prescription order through the interface 220, the data transfer unit 211 can execute a function of transmitting the order to the prescription transfer system 300.

The cache storage unit 212 is a component that temporarily stores medical information about a patient transmitted from the database 110. Accordingly, the processor 210 can extract and process information using medical information stored in the cache storage unit according to settings through the interface or a request of a user. In addition, when a user inputs medical information, the processor 210 can reflect the input medical information in medical information stored in the cache storage unit and transmit the medical information stored in the cache storage unit 212 to the database 110 such that medical information in the database can be updated.

The object generation unit 213 is a component that generates an object to be displayed in each section 221 of the interface 220 among medical information about a patient transmitted from the database 110. Some of medical information about a patient transmitted from the database 110 are displayed in a section of the interface and various display formats may be provided according to fields. Accordingly, the processor 210 can extract information by displayed dates and information about fields from medical information stored in the cache storage unit 212 and the object generation unit 213 can generate an object to be displayed in a section for each date and each field in a display format defined for each field.

For example, results with respect to blood examination in the examination field may include various types of information as displayed in a detailed query window, as illustrated in FIG. 4. However, a section corresponding to a blood examination item is defined such that only four major items among the blood examination results are displayed in a designated pattern. Accordingly, the object generation unit 213 generates an object such that only corresponding information can be extracted from blood examination result information and displayed in a designated pattern.

An object generated by the object generation unit 213 can be realized in various form according to the field corresponding thereto. As illustrated in FIG. 4, objects can be generated such that they are differently displayed in chart forms defined according to examination types such as blood examination and basal metabolism profile examination. In addition, a result such as an X-ray imaging result among examination results may be generated such that it is displayed as an image file on a section and a result such as an ultrasonic examination result may be generated such that it is displayed as a moving image reproduced on a section. Further, the diagnosis field and the medical record field may be configured to be displayed in a text form. In this manner, the object generation unit can generate an object for each field and each section scale which will be described later in various forms.

Meanwhile, the input processing unit 214 is a component that processes details input by a user through the interface. The user can perform an input operation such as inputting a diagnosis, a medical record, or prescription corresponding to a reference date through the interface. Details input by the user through the interface in this manner are recognized by the input processing unit, stored in the cache storage unit 212 and processed such that they can be updated to the database 110 or processed to be transmitted to the prescription transfer system 300. Then, the processed information can be transmitted to the database or external components including the prescription transfer system through the data transfer unit 211.

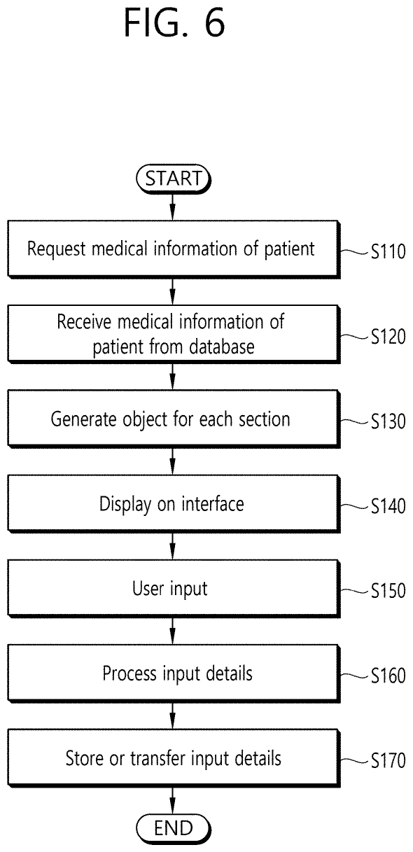

Operations performed by the processor 210 in the medical information query and input system have been described above focusing on major components. FIG. 6 is a flowchart illustrating a medical information query and input method performed by the system of FIG. 1. Hereinafter, each step will be described in detail with reference to FIG. 6.

First, a step in which a user requests medical information about a patient through the interface is performed (S110). This step may be performed in such a manner that the user inputs the name or ID of the patient through the screen of the interface 220.

The processor 210 retrieves the requested medical information about the patient from the database 110 (S120). As described above, the database 110 may be a database included in a user terminal, a database included in an additional server device, or a cloud server, or a database included in an additional examination device, another medical institution or a device carried by a patient. This step may be performed in such a manner that the medical information is received through the data transfer unit 211 of the processor, and the received medical information may be stored in the cache storage unit 212.

The processor 210 performs a step of generating an object that can be displayed in each section on the interface using the medical information about the patient transmitted from the database (S130). In this step, the processor extracts target information among the medical information about the patient on the basis of set values (e.g., displayed date periods, selected field information, etc.). Here, details included in the target information may be classified by dates and fields (by subfields when there are subfields) in units corresponding to respective sections. The object generation unit 213 generates each object by processing information corresponding to each classified section in a defined form according to the field or subfield corresponding thereto. Here, the defined format may be different according to field types and may be different according to scales set by the user.

Objects generated by the processor 210 are displayed through respective sections of the interface 220 (S140). As described above, details displayed in each section can be generated in various forms such as a text form, a chart form, an image form, and an image reproduction form according to fields or subfields. Accordingly, the user can simultaneously check pieces of information by dates through the interface in a time series and check information by classifying the information by fields according to characteristics thereof.

Meanwhile, the user performs a step of inputting necessary details through the interface 220 while querying the medical information about the patient through the interface 220 (S150). The input details may be a medical record, prescription, or details with respect to a medical reservation or an examination reservation. A specific input method will be additionally described below.

When the user inputs the aforementioned details, the processor 210 processes the input information according to an input field and details (S160). For example, when the user inputs information such as a medical record, the input processing unit 214 processes the information such that this information can be added to existing medical information about the patient stored in the cache storage unit 212 and stored therein. Alternatively, when the user inputs a prescription order or reservation information, the processor 210 generates a command for transferring the corresponding information to the prescription transfer system 300. In addition, the processor 210 can process information input by the user through a method defined according to characteristics of the information.

When processing of the input information is performed in this manner, the processor 210 performs a step of storing the input details or transferring the input details to another component (S170). This step can be performed through the data transfer unit 211 of the processor 210. In this step, content of the cache storage unit storing the medical information about the patient in which the input details have been reflected is transmitted to the database in which the previous medical information about the patient is stored such that the medical information about the patient can be updated alternatively, the input details can be transmitted to the prescription transfer system 300 or a device corresponding to the input details.

### Input of Medical Information

Information input by a user through the interface may be largely classified into two. The first one is a field (hereinafter referred to as a first field) through which determination, an opinion and a medical examination by interview of a user, and the like are input, such as a diagnosis field, a medical record field, and a referral field. This is generally input using text and input as medical information corresponding to a reference date in many cases. The other one is a field (hereinafter referred to as a second field) through which a user makes a reservation of examination or rehabilitation on a reference date or after the reference date or inputs a prescription order such as medicine, medical treatment, examination, or rehabilitation, such as an examination field and a prescription field. Hereinafter, methods of inputting details by a user according to characteristics of these fields will be described in detail.

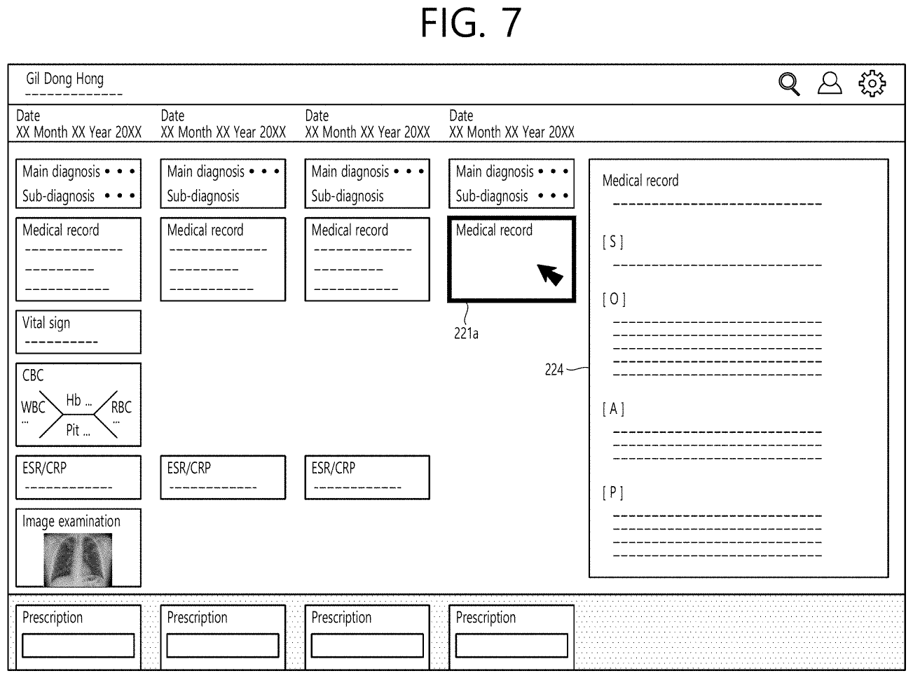

FIG. 7 is a diagram illustrating an example of inputting details of the first field to the interface (first input method). When medical information about a patient is queried, information prior to a reference date is displayed on the interface in a section format but information corresponding to the reference date and dates after the reference date exists as a blank region unless reserved information is present. Here, if a user selects a blank position of a corresponding field (medical record field) in a reference date column in order to input a medical record corresponding to the reference date during a medical treatment, a vacant section 221a is generated at the corresponding position, if the generated vacant section 221a is selected, an additional input window 224 is displayed on one side of the interface like the aforementioned detailed query window. Accordingly, when the user inputs details of the medical record through the input window 224 and closes the input window 224, an object corresponding to the medical record field of the reference date is generated through the processor and displayed in the corresponding section. The step of generating a vacant section when a blank position is selected may be omitted according to an embodiment. Further, when a blank position is selected, the input window may be immediately displayed when an additional mark (e.g., “+”) indicating addition of a section is displayed and an object reflecting the corresponding details may be generated upon completion of input and a section may be generated at the corresponding field position.

Meanwhile, in a case where a patient frequently visits a medical institution for a specific period of time because of the same lesion, symptoms of the patient may be similar and doctors may have similar opinions in many cases. Accordingly, the interface includes a function of using information input on a previous date instead of newly inputting all details when the first field is input.

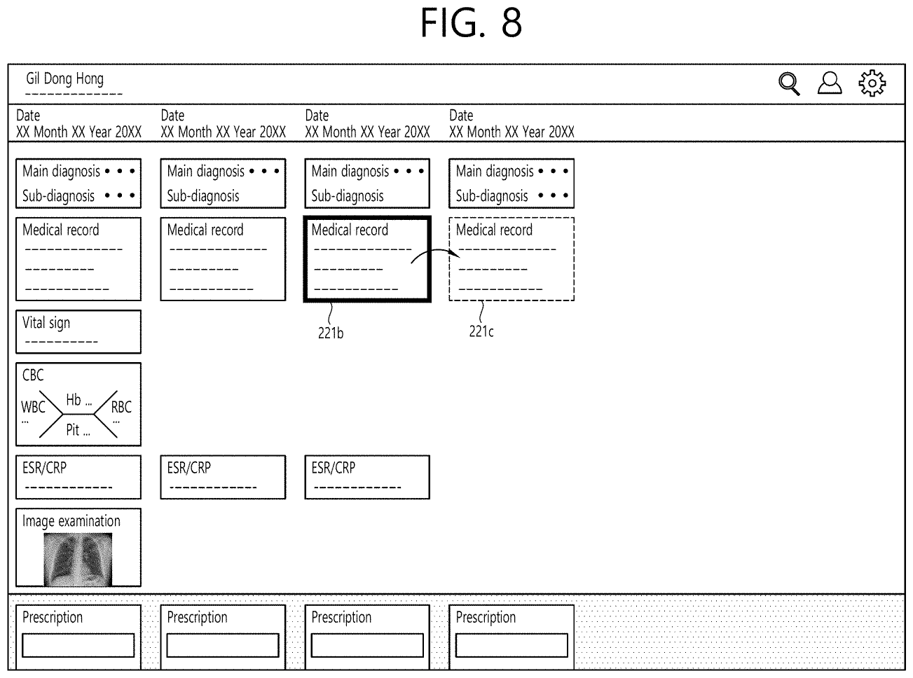

FIG. 8 is a diagram illustrating another example of inputting the details of the first field to the interface (second input method). When a user intends to input medical information corresponding to a reference date using medical information before the reference date, the user can select a section prior to the reference date which will be used and move the selected section to a corresponding field position of the reference date. This operation may be performed through a conventional drag-and-drop method. A section 221c having the same details as those of an existing section 221b is generated in the same field of the reference date through the aforementioned operation. This corresponds to a state in which the same details as the existing section have been input to the corresponding field of the reference date (this can be used in the case of the same diagnosis). In addition, when the user selects the section 221c generated on the reference date, the input window 224 is displayed on one side, as illustrated in FIG. 7. Here, the input window 224 is in a state in which the same medical information as that corresponding to the existing section 221b has been input, and the user can correct (delete, substitute, or add) the existing details through the input window 224. When the input window 224 is closed, an object corresponding to the corresponding field of the reference date can be regenerated and displayed in the corresponding section 221c.

As described above, according to the present embodiment, user convenience can be improved by performing an input operation using existing medical information and the input operation can be applied to various terminal devices using a method of dragging and dropping a section. Here, movement of an existing section may be limited within the same field in order to prevent improper input.

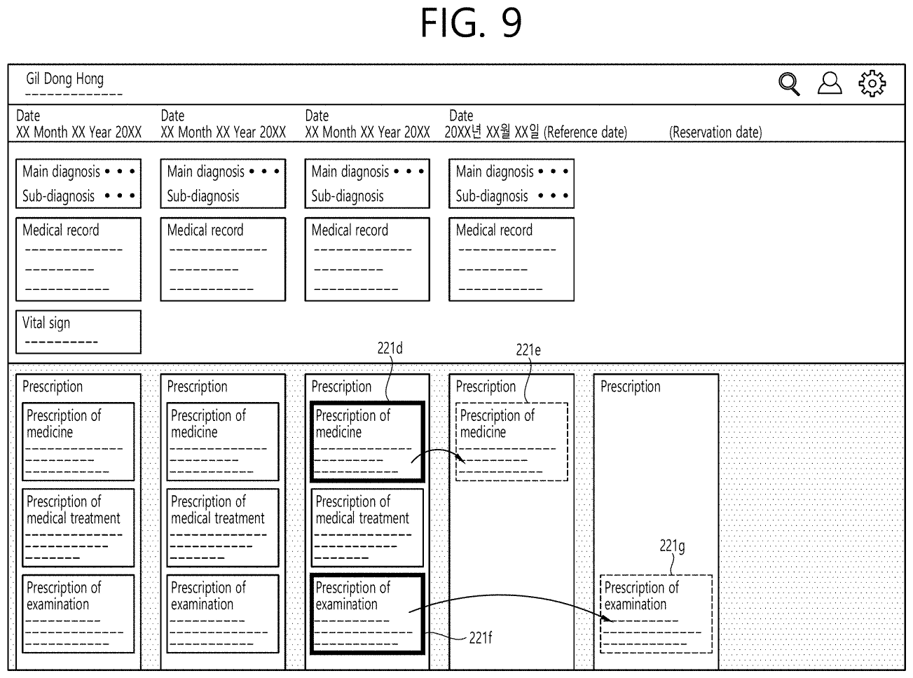

FIG. 9 is a diagram illustrating an example of inputting details of the second field to the interface. As described above, the second field such as the examination field or the prescription field requires a function of inputting various orders and reservation information on a reference date or after the reference date. In particular, since details of various prescription orders are highly likely to be similar to prescription performed prior to the reference date, input can be performed using information prior to the reference date as in the aforementioned second input method.

Specifically, FIG. 9 shows prescription field windows and illustrates a state in which windows hidden at the bottom of the interface are selected by a user to rise up. The prescription field may include sub-prescriptions such as prescription of medicine, prescription of medical treatment and prescription of examination. The user can input prescription orders to prescription field windows using the aforementioned second input method.

For example, when the user intends to input a medicine prescription order, the user selects a past section 221d of the medicine prescription field in which a similar prescription is performed and moves the section 221d to a reference date position. Accordingly, a prescription order can be input in such a manner that a section 221e generated at the reference date position is selected and details are corrected. Furthermore, in the case of a medical treatment prescription, a section corresponding to the same medical treatment prescription in the past can be moved to the reference date position and an examination prescription order can be input.

Furthermore, in the case of some such fields that require a reservation function (e.g., prescription of examination and prescription of rehabilitation), a section may be moved to a position after a reference date instead of being moving to the reference date and reservation input may be performed. For example, when examination reservation is required, a past section 221f in which the same examination was prescribed can be moved to a position after the reference date (to the right side of the reference date column) in a prescription field window and the corresponding examination can be reserved. Here, a new section 221g is generated at a position of the corresponding subfield after the reference date and may include even a function of selecting a reservation date if required. In this case, a reservation date can be displayed on the time axis at a position at which a section corresponding to a reservation is generated.

As described above, in the case of the second field, the position of an existing section can be moved, a prescription order can be input and details of reservation can be input through the second input method. Although FIG. 9 mainly illustrates a process of inputting through a prescription field window, a prescription order, and a reservation may be input in the same manner in the examination field. For example, when a user intends to input an examination prescription or an examination reservation in a state in which prescription field windows have not risen up, it is possible to move a section corresponding to the corresponding examination among sections displaying past examination results in the examination field to the reference date or a position after the reference date and input the examination prescription or the examination reservation.

As described above, although movement of a section may be limited within the same field or the same subfield in the second input method, it may be exceptional in examination related fields. Specifically, in the examination field and an examination prescription field of the prescription field, if a user prescribes or reserves a specific examination using one of the fields, a corresponding section can also be generated in the other such that details of prescription or reservation can be checked. Accordingly, the user can prescribe or reserve additional examination without a prescription field window rising up while checking previous examination results through the examination field. In addition, a section generated according to examination prescription or reservation can be displayed to be distinguished from sections displaying previous examination results because examination result information corresponding thereto does not exist yet. For example, while a section displaying results of ultrasonic examination performed before the reference date is displayed such that a moving image is reproduced, a section generated according to examination prescription or examination reservation can be displayed such that an object indicating a vacant section or absence of examination information is displayed. Accordingly, the user can also check whether examination reserved on the reference date at the time of past medical treatment has been performed through a section displayed in the examination field.

Input of details of prescription or reservation to the second field using the second input method has been described above. However, the second field input method is not limited thereto and may also include an input function using the first input method of generating a vacant section at a position on the reference date or after the reference date and directly inputting information.

### Display According to Medical Information Query Section Adjustment

The interface no according to the present embodiment may further include a function of setting and controlling time axis information by a user. Here, the time axis information may be a date section displayed on the screen of the interface among medical information about a patient or the number of pieces of displayed information for each date. A user can query a medical history of a patient microscopically or macroscopically by controlling the time axis information as necessary. Here, the size of the section 221 displayed on the interface 220 and an object displayed in the section also change according to time axis setting information. Details information is displayed through a section having a relatively large size when a small number of pieces of information for each date is requested, whereas a section size may decrease and information displayed through a section may be simplified when a large number of pieces of information for each date is requested. In this manner, a section and details of an object displayed in the section can be displayed in different scales according to set time axis information.

For example, sections and objects displayed in the sections are divided into a first scale, a second scale and a third scale according to time axis information in the present embodiment. The first scale is a form displayed when a displayed date period is short or the number of displayed dates is small, and the second scale and the third scale are forms displayed when a displayed date period is longer and the number of displayed dates is larger. First, a section size is largest in the first scale and becomes smaller in the second scale and the third scale. In addition, each object can be configured to display most detailed information the first scale and display only a small amount of simple information in the second scale and the third scale. Accordingly, when the time axis information is changed by user settings, the processor 210 can determine an object scale according to the time axis information, generate an object in the determined scale and display the a object in a section. That is, even the same medical information can be displayed differently on the interface accord axis information.

The processor 210 can define a display format depending on each scale according to each field and each subfield. As time axis setting information changes details displayed in each section may change differently for respective field or subfields.

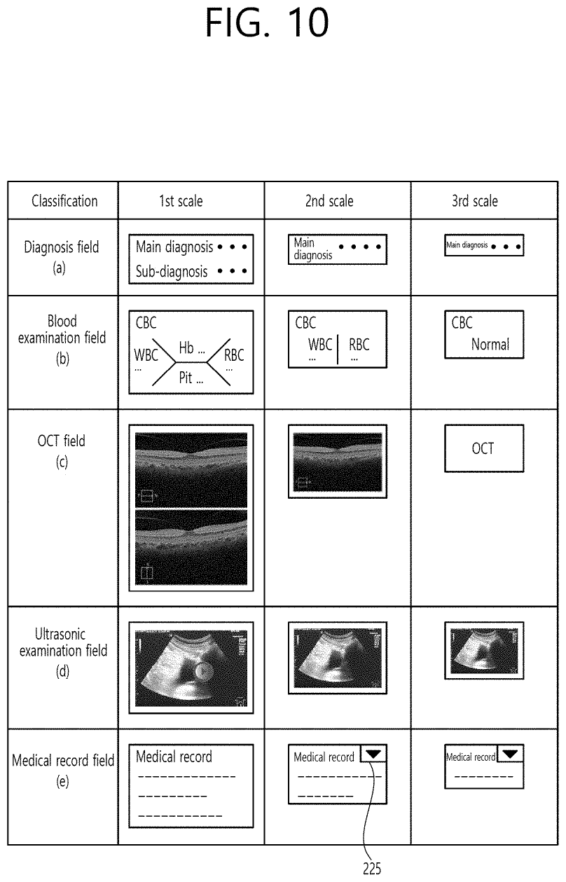

FIG. 10 is a diagram illustrating display formats according to scales for each field or each subfield. In FIG. 10, a indicates a diagnosis field, b indicates a blood examination field, c indicates an X-ray examination field, d indicates an ultrasonic examination field and e indicates a medical record field.

First, information of the diagnosis field may be configured such that both a main diagnosis and a sub-diagnosis are displayed in the first scale as d only the main diagnosis is displayed with the stale-diagnosis omitted in the second scale and the third scale.

In addition, information of the blood examination field may be configured such that friar pieces of major information are displayed in a chart form in the first scale, only two pieces of major information to priority is assigned are displayed in the second scale, and only a determination result such as “normal” or “abnormal” according to an examination result is displayed in the third scale.

Furthermore, the OCT examination field may be configured to display a whole examination image in the first scale, to display only art of image information to which priority is assigned among whole examination information in the second scale, and to display a thumbnail image predetermined the corresponding subfield in the third scale.

Moreover, the ultrasonic examination field may be configured to display a moving image reproduced through a section in the first scale (a specific slide is displayed in the figure because it is difficult to display moving image reproduction), to display a specific slide image to which priority is assigned in the second scale, and to display a thumbnail image predetermined for the corresponding subfield in the third scale.

However, some fields or some subfields may be configured such that details of an object displayed through a section are maintained even when a section size changes according to time axis setting information. This is because, in the case of some information such as medical records, a User needs to check whole information even when a scale changes. As illustrated in (e) of FIG. 10, some fields including the medical record field may be configured such that the same details can be displayed through a corresponding section using a scroll bar or a window opening function even when a section size decreases as a scale changes.

As described above, the processor can determine a scale on the basis of time axis information requested by a user, generate a section and an object through a method defined according to a field or a subfield and display the section and the object on the interface. Although FIG. 10 illustrates an example of three-stage scales, the present invention is not limited thereto and display formats may be configured to change to scales of four or more stages as necessary.

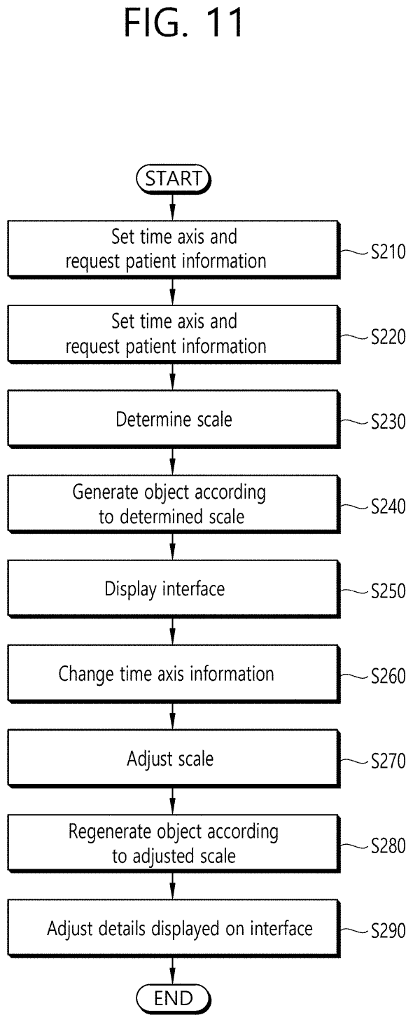

FIG. 11 is a flowchart illustrating an interface display method according to time axis adjustment. The interface display method according to time axis adjustment will be described in detail with reference to FIG. 11.

First, a step in which a user sets an initial use environment of the interface 220 is performed (S210). The set initial use environment may include initial time axis information. In addition, the user may request information about a specific patient through the interface 220 (S220).

When the patient information is requested, the processor 210 determines a scale of a section and an object displayed on the interface 220 in consideration of the initial time axis information and medical information about the patient provided from a database (S230).

Although the first scale can be determined in general, the second or third scale may be determined when a large amount of date information needs to be displayed among the requested patient medical information.

When the processor 210 determines a scale, an object to be displayed in a section is generated on the basis of the determined scale (S240). Here, an object form is defined according to each scale for each field or each subfield, and the processor generates the object in the object form. Furthermore, the processor may assign priority to specific information, image, or slide among medical information with respect to a corresponding field through a predefined method and generate an object according to each scale. An object generated in this manner is displayed in a section having a size adjusted in the corresponding scale on the interface (S250).

Meanwhile, the user can change time axis information such as a displayed date period or the number of displayed dates through the interface (S260). When there is such a request for change. the processor 210 executes a step of adjusting the scale in consideration of the medical information about the patient and the changed time axis information (S270). In addition, the processor regenerates an object for each field in the adjusted scale according to predefined details (S280) and displays the object through each readjusted section of the interface (S290). In this manner, the interface according to the present embodiment can cope with a time axis change request of the user.

Hereinafter, display on the interface according to the above-described time axis change will be described through several examples. As described above, the interface according to the present embodiment is configured such that a date period in which medical information about a patient will be queried can be selected. For example, a date period can be adjusted to three periods: one month (a first query period); six months (a second query period); and three years (a third query period) in the present embodiment.

Here, the processor 210 can be configured to determine a scale of a section and an object according to a type of a set date period. For example, the first scale can be determined when the set date period is the first query period, the second scale can be determined when the set date period is the second query period, and the third scale can be determined when the set date period is the third query period. However, in this configuration, when little medical history, such as visit to medical institutions and prescriptions for the past six months, exists even if the second query period is set, there is a disadvantage that displayed medical information is the same as that when the first query period is set but a display scale decreases.

Accordingly, the processor 210 can extract medical information of a corresponding query period according to the type of a set date period and determine a scale on the basis of the number of pieces of information for each date (the number of dates) which need to be displayed on the interface. For example, the first scale can be determined if the number of pieces of information for each date is less than n1, the second scale can be determined if the number of pieces of information for each date is equal to or greater than n1 and equal to or less than n2, and the third scale can be determined if the number of pieces of information for each date exceeds n2. In this case, even if the user increases a query period, medical information can be displayed on the interface in a suitable scale in response to the number of pieces of information about a patient for each date which is actually displayed on the interface.

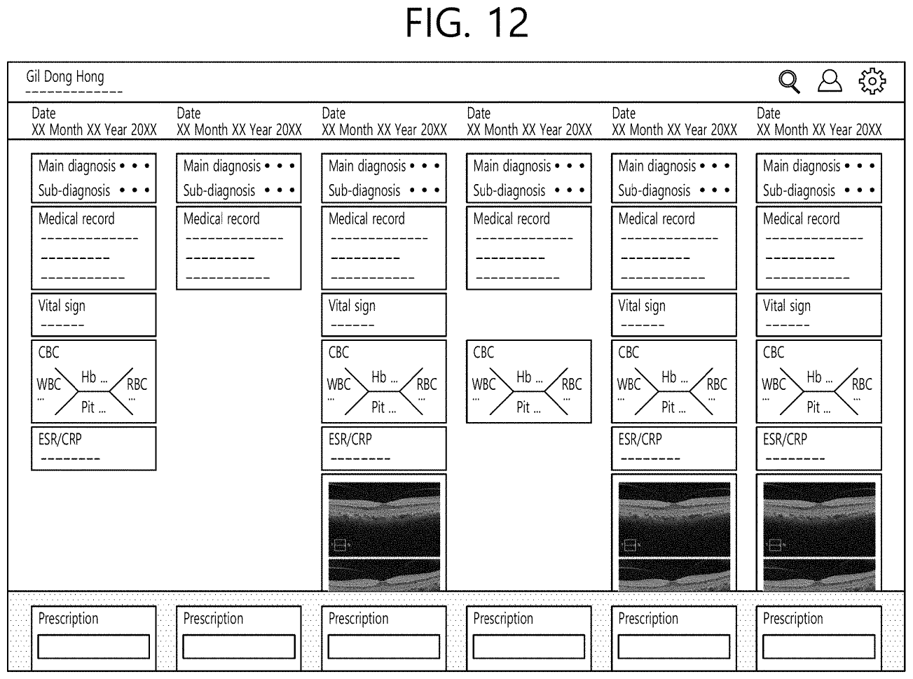

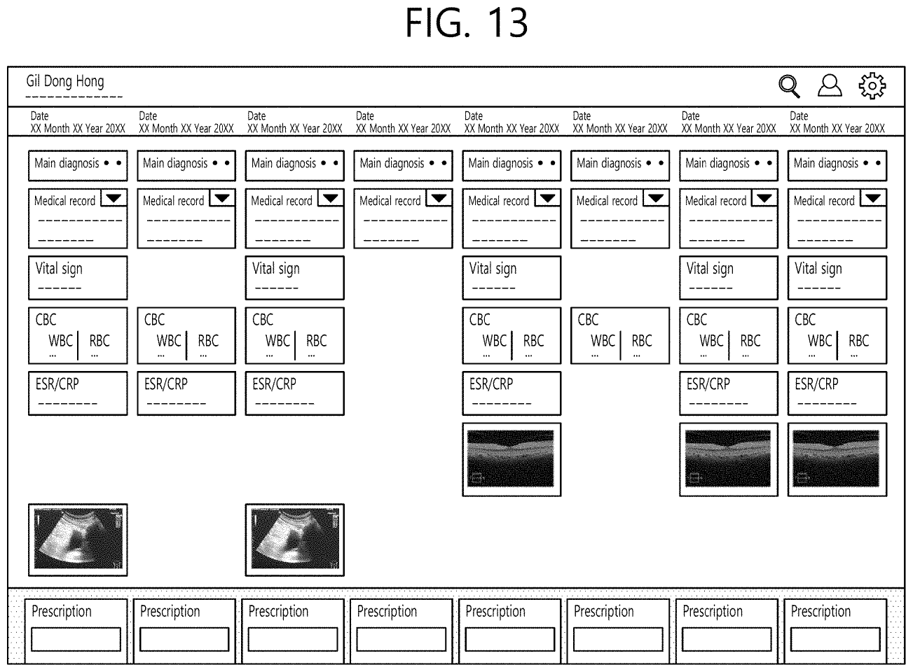

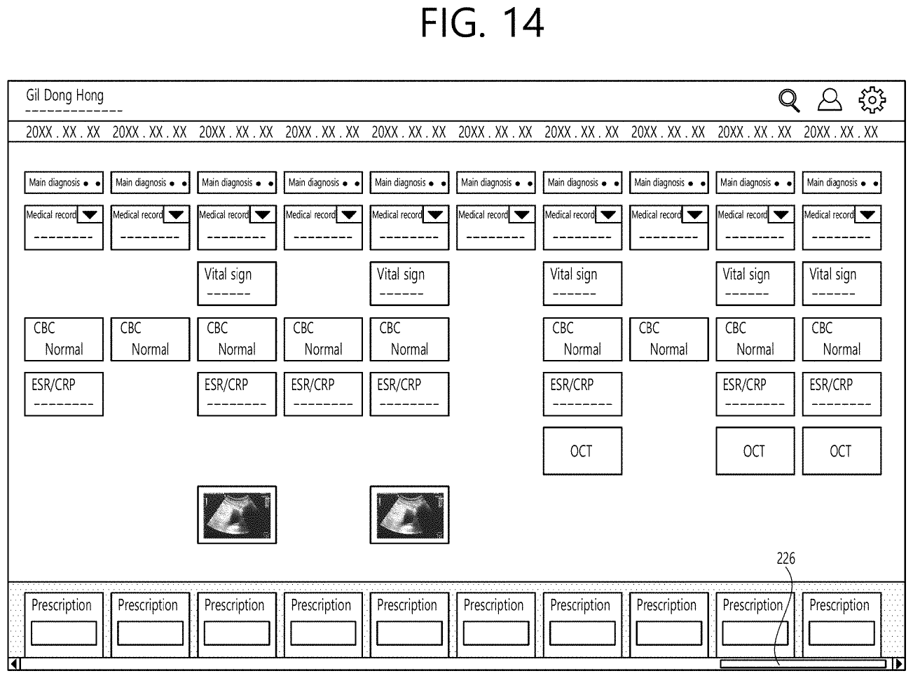

FIG. 12 is a diagram illustrating the interface in a state in which the first query period is set, FIG. 13 is a diagram illustrating the interface in a state in which the second query period is set, and FIG. 14 is a diagram illustrating the interface in a state in which the third query period is set.

As illustrated in FIGS. 12 to 14, when the first query period is set, a user can query recent medical record information about a patient in more detail. In addition, when the second query period and the third query period are set, it is possible to ascertain and diagnose a state of a patient from a macroscopic view by querying a past medical history of the patient as well as a recent medical history in a time series.

However, when the number of pieces of information about a patient for each date which need to be displayed increases since a long query period is set, it may be difficult to display the information on the interface screen. In this case, a fourth scale and a fifth scale may be added to reduce the amount of displayed details of information, but it is difficult fora user to identify information if the amount of displayed information is excessively reduced. Accordingly, the processor may be configured to have a reference value for the number of pieces of information for each date which can be displayed on the interface in a set minimum scale (the third, scale in the present embodiment) and to horizontally move the interface window using a scroll bar 226 or the like when the number of pieces of information for each date exceeds the reference value. In this case, whole information can be displayed in a form that can be easily identified by a user even when there is a large number of pieces of information for each date which need to be displayed.

Although the interface is configured such that one of the first to third query periods provided as options is selected by a user in the above-described present embodiment, the present invention is not limited thereto. For example, the interface may be configured such that the time axis is linearly controlled through a method of magnifying/reducing the interface screen (e.g., scroll up/down of a mouse) to sequentially control the number of pieces of information for each date displayed on the time axis. In this case, it is possible to provide a desired query environment of a user by freely adjusting a query period.

### Selective Query of Information Relevant to Selected Diagnosis

The interface according to the present embodiment includes a function of extracting only medical information related to a representative value that a user desires from among various types of medical information about a patient and displaying the extracted medical information.

When a patient visits a medical institution because of various lesions, medical information about the patient displayed on the interface may include diagnosis, examination, and prescription records with respect to the various lesions. Accordingly, when a user intends to query information related to a specific diagnosis, the user needs to sort information because pieces of less relevant information are mixed and the efficiency of query deteriorates because the amount of information displayed on the screen is limited. In the present embodiment, when a user selects a diagnosis as a representative value, only medical information related to the diagnosis can be extracted and displayed and thus query convenience of the user and the efficiency of query can be improved.

To this end, the interface includes a function of selecting a representative value b a user. For example, a user can select a representative value by selecting a section 221h of a diagnosis field in which a diagnosis of interest is displayed. Alternatively, the user can select a representative value by inputting a diagnosis of interest through an additional keyword input window. Although a diagnosis will be illustrated as a representative value below, the present invention is not limited thereto and another item such as a medicine prescription item, a medical treatment prescription item, or an examination prescription item may be selected as a representative value.

The processor 210 stores data that is a standard for determination of relevance with respect to each representative value selectable by a user. Accordingly, when a user selects a representative value, related information is extracted from among medical information about a patient stored in the cache storage unit 212 according to the relevance determination standard. Accordingly, the object generation unit 213 can exclude less relevant information, generate an object only for the extracted information and display the object in a section on the interface 220.

Here, information extracted in the processor 210 may be information corresponding to a specific field or a specific subfield. For example, when a diagnosis with respect to a retinal disease is selected as a representative value, the processor can extract an intraocular pressure examination field, an eyeground OCT examination field and a blood examination field relevant to the diagnosis from among medical information about the patient and exclude a pulmonary function test field and an ultrasonic examination field irrelevant to the diagnosis.

Furthermore, the processor 210 can extract a specific field or a specific subfield and also extract highly relevant detailed information from among information included in the extracted field. In addition, the object generation unit 213 can generate an object such that the highly relevant detailed information is displayed on the interface. Here, the object generation unit 213 can generate an object through a method defined differently from a method of displaying information OD the default interface screen. For example, when a diagnosis with respect to diabetes is selected as a representative value, the blood examination field ma be extracted as a relevant field and an object may be generated such that a blood sugar level, a lipid level, and the like among blood examination information are displayed in a section.

When such a selective display method considering a degree of interest of a riser is employed, date information displayed on the interface may be changed from information displayed on the interface before the user selects a representative value. This it, because information of a date when a medical treatment for a disease different from the representative value has been performed is irrelevant and thus can be excluded. Further, only relevant fields among information of displayed dates are selectively displayed. Moreover, pieces of information can be displayed differently on the basis of specific relevant information even when the pieces of information correspond to the same field.

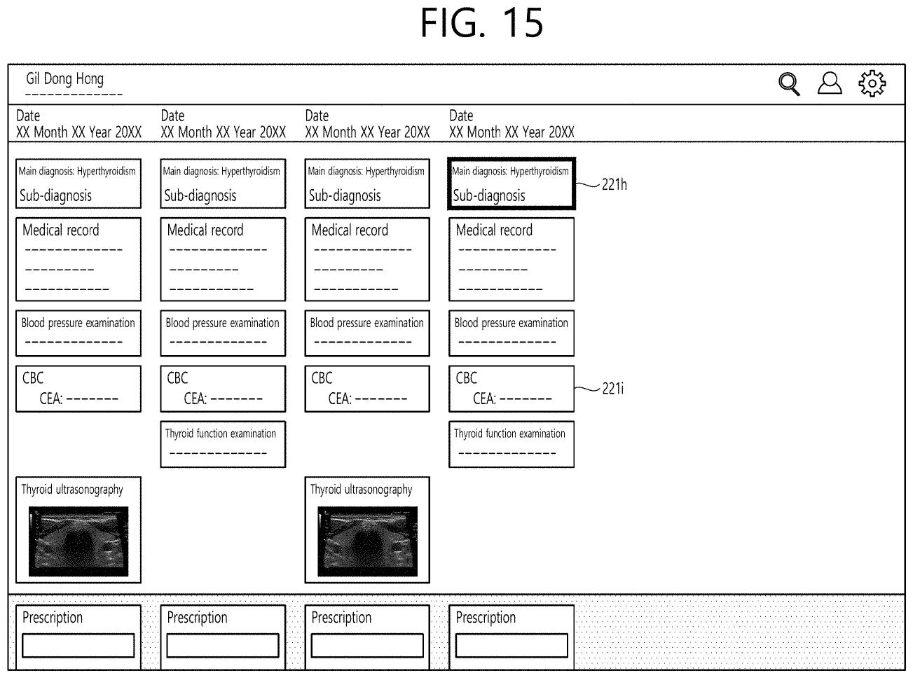

FIG. 15 is a diagram illustrating details displayed on the interface when a predetermined representative value is selected. The interface illustrated in FIG. 1 is displayed when a user selects a diagnosis with respect to hyperthyroidism as a representative value.

As medical information to be displayed on the interface, only dates on which hyperthyroidism was diagnosed and medical treatments were performed are extracted from among whole medical information about a corresponding patient and displayed. However, a date including examination information highly relevant to the representative value can be displayed on the interface although a diagnosis corresponding to the date is different from the representative value.

In addition, fields relevant to hyperthyroidism among information of the corresponding dates are mainly displayed on the interface. However, a diagnosis field and a medical record field among the information of the corresponding dates can be displayed on the interface irrespective of a degree of relevance to the representative value. Specifically, results of blood pressure examination, blood examination, thyroid function examination, and thyroid ultrasonography that are highly relevant to hyperthyroidism among examination fields are displayed on the interface, as illustrated in FIG. 15. The interface may be configured such that blood examination results among the aforementioned results are displayed focusing on CEA values highly relevant to hyperthyroidism differently from the manner displayed in FIGS. 3 to 5 (refer to 221i),

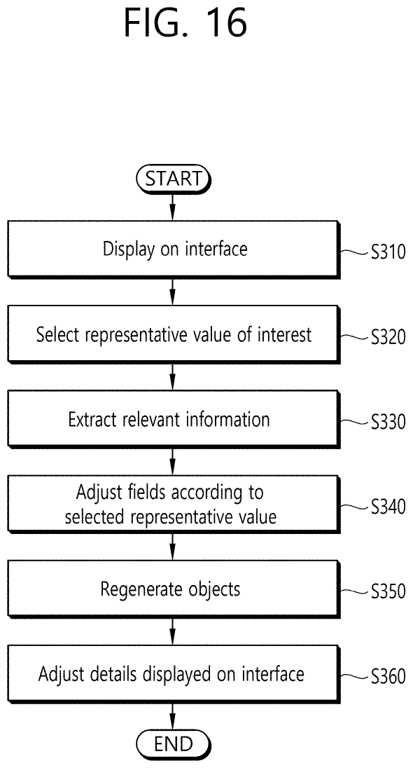

FIG. 16 is a flowchart illustrating a method of selectively displaying information related to a representative value selected by a user. Hereinafter, the method of displaying information related to a representative value will be described in more detail.

First, a step in which medical information about a patient requested by a user is received from a database and displayed on the interface is performed (S310). Specific description of this step is substituted with the above description of S110 to S140 of FIG. 6.

The user performs a step of selecting a representative value of interest through the interface (S320). Here, the representative value may be a diagnosis of the patient or various items such as a specific prescribed medicine and a prescribed medical treatment.

When the user sets a representative value through the interface, the processor performs a step of extracting information relevant to the representative value from among medical information about the patient (S330). As described above, the processor 210 stores a standard for determination of relevance to each representative value and the processor 210 extracts relevant information from among medical information about the patient stored in the cache storage unit 212 on the basis of the standard.

In addition, a step of adjusting fields displayed on the interface may be performed (S340). In this step, displayed field items and the order thereof can be determined according to presence or absence of information about corresponding fields among the medical information extracted in the previous step. However, field arrangement displayed on the interface according to a selected representative value may be predefined irrespective of extracted medical information and fields may be adjusted as defined. Further, dates displayed on the time axis may also be adjusted such that information for each date including irrelevant information is excluded and information for each date including relevant information is displayed according to extracted information. In addition, the processor can configure each section in the interface according to the adjusted fields and date information. Here, each section displayed on the interface is generated according to the adjusted fields and date information and can be generated at a position at which information corresponding to a field of a corresponding date among the extracted information exists. However, thi, step is not essentially performed as a separate step, and date information and field information may be automatically adjusted as results of execution of other steps.

The object generation unit 213 regenerates objects for displaying the extracted information in each section of the interface (S350). Here, an object generation method may be defined differently according to selected representative values. For example, even if objects with respect to the same field are generated, the objects may be regenerated differently such that detailed information highly relevant to a representative value among information about the field can be directly displayed to a user (for example, a highly relevant numerical value among blood examination results is displayed).

The regenerated objects are displayed in each section of the interface and thus details displayed on the interface can be adjusted (S360). Through this step, information relevant to the representative value selected by the user can be classified into sections and displayed on the interface in a time series.

As described above, according to the present embodiment, a user can selectively display only information relevant to a representative value of interest, such as a specific diagnosis, on the interface and query the information.

The medical information (piety and input method according to the above-described embodiment of the present invention may be implemented as a program (including an application) to be executed in association with a user terminal and stored in media.

This program may include code encoded in a program language readable by an arithmetic unit of a terminal in order for a user terminal to read the program and execute the above-described methods implemented in the program. This code may include functional code associated with functions that define functions necessary to execute the above-described methods and may include execution procedure related control code necessary for an arithmetic unit of a terminal to execute the aforementioned functions according to a predetermined procedure.

In addition, media in which the program is stored are not media storing data for a moment, such as a register, a cache, and a memory, and are media that semipermanently store data and are readable by devices. Specifically, examples of the media include a ROM, a RAM, a CD-ROM, a magnetic tape, a floppy disk, and an optical data storage device, and the media are not limited thereto. For example, the aforementioned program may be stored in various recording media on various web servers accessible by user terminals. Furthermore, the media may be distributed to computer systems connected via a network and computer readable code may be stored therein in a distributed manner.

Although an embodiment of the present invention has been described in detail, the present invention is not limited to the above-described embodiment. Those skilled in the art will appreciate that various modifications and variations can be made in the present invention without departing from the spirit or scope of the invention described in the appended claims.

1. A medical information query and input method comprising:
retrieving, from a database, medical information about a patient before a reference date;
generating, from the medical information about the patient, objects for each date according to a set field item; and
displaying, on an interface, each of the objects in a separate section format, by date in a time series in a corresponding field.

2. The medical information query and input method of claim 1, further comprising inputting details of a field corresponding to the reference date or a date after the reference date using a section displayed on the interface prior to the reference date.

3. The medical information query and input method of claim 2, wherein the inputting comprises selecting an already displayed section of the corresponding field and moving the section through a method of dragging the section to a position of the reference date or a position after the reference date to generate a new section at the corresponding position.

4. The medical information query and input method of claim 3, wherein the inputting comprises:
generating a new section by dragging a section of a predetermined field to be input before the reference date to the position of the reference date; and
generating medical information corresponding to the predetermined field of the reference date.

5. The medical information query and input method of claim 4, wherein the generating of the medical information comprises selecting the generated section by a user to call an input window and correcting medical information of an existing section displayed through the input window to generate the medical information corresponding to the predetermined field of the reference date.

6. The medical information query and input method of claim 5, wherein the generating of the medical information comprises regenerating an object in which details corrected by the user have been reflected and displaying the regenerated object in the generated section.

7. The medical information query and input method of claim 4, wherein the predetermined field is one of a diagnosis field, a medical record field, and a prescription field.

8. The medical information query and input method of claim 3, wherein the inputting comprises:
generating a new section by dragging a section of a predetermined field to be reserved at a position corresponding to the reference date or before the reference date to the position of the reference date or a position after the reference date; and
generating reservation information corresponding to the predetermined field after the reference date.

9. The medical information query and input method of claim 8, wherein the generating of the reservation information comprises selecting the generated section by a user and inputting a reservation date by the user.

10. The medical information query and input method of claim 8, further comprising transmitting the generated reservation information to a prescription transfer system.

11. The medical information query and input method of claim 8, wherein the predetermined field is one of an examination field and a prescription field.

12. The medical information query and input method of claim 8, wherein, when an examination result corresponding to the reserved information does not exist in the medial information about the patient transmitted from the database, the examination result is displayed through the generated section using a predefined method such that absence of the examination result is able to be recognized.

13. The medical information query and input method of claim 1, wherein, when a user selects a position at which a section is not displayed on the interface, an input window corresponding to a corresponding field of a corresponding date is displayed, and a new section in which an object corresponding to details input by the user through the input window is displayed is generated at the position.

14. The medical information query and input method of claim 1, wherein, when the user selects the section, detailed information corresponding to the section is displayed through an additional detailed display window.

15. The medical information query and input method of claim 1, wherein, when the user selects a specific field, only medical information corresponding to the selected field is extracted and displayed through an additional window in a time series.

A medical information query and input program executing:
retrieving, from a database, medical information about a patient before a reference date;
generating, from the medical information about the patient, objects corresponding to set time axis information and set fields; and
displaying, on an interface, each of the objects in a separate section format in a time series according to a corresponding field.

17. A medical information query and input system comprising:
an interface for displaying medical information about a patient in separate Section formats according to date information and field information and allowing a user to input medical information about a patient; and
a processor for processing medical information about a patient provided from a database such that the medical information is able to be displayed on the interface and processing information input through the interface,
wherein the processor includes:
a data transfer unit for transmitting/receiving medical information about a patient to/from a database;
an object generation unit for generating, from medical information about a patient provided from the database, each object corresponding to set time axis information and a set field such that the object is able to be displayed in a section on the interface; and
an input processing unit for processing information input by a user through the interface.
